(19) United States
(12) Patent Application Publication
(10) Pub. No.: US 2015/0161535 A1
(43) Pub. Date: Jun. 11, 2015

(54) SYSTEMS AND METHODS FOR MAPPING MEDICAL GUIDELINES TO CLINICAL WORKFLOWS AND LOGISTICAL OPERATIONS

(71) Applicant: Gary and Mary West Health Institute, La Jolla, CA (US)

(72) Inventors: Amnon Ptashek, San Diego, CA (US); Erik Tyrrell-Knott, Del Mar, CA (US); Vikas Tandon, San Diego, CA (US); Joseph Martin Smith, Rancho Santa Fe, CA (US); Scott Edward Cassidy, Phoenix, AZ (US)

(73) Assignee: Gary and Mary West Health Institute, La Jolla, CA (US)

(21) Appl. No.: 14/099,624

(22) Filed: Dec. 6, 2013

(51) Int. Cl. G06Q 10/06; G06Q 50/22

(52) U.S. Cl. CPC G06Q 10/0633; G06Q 50/22

(57) ABSTRACT

A system for automating coordinated care management by mapping medical guidelines to clinical workflows and logistical operations may be provided. The system may include a mapping system, a medical guidelines database, a clinical workflow database and a logistical workflow database. The mapping system may receive medical guidelines from the medical guidelines database, generate clinical workflows to be stored in the clinical workflow database, and generate logistical workflows to be stored in the logistical workflow database. The logistical workflows can be generated from clinical workflows or directly from medical guidelines. The system may include a communications system that automatically manages communications between participants in a healthcare system using computer code generated based on the logistical workflows. The system may also monitor patient response and outcome information in order to yield predictive patient engagement efficiencies to address current fragmentation and inefficiencies of care coordination.

### BACKGROUND

Healthcare systems that have typically provided care to patients on a fee-for-service basis are increasingly emphasizing accountable care in which portions of the healthcare system seek to monitor and guide the patient's condition and activities while the patient is outside of the healthcare environment. For example, the blood pressure, blood glucose levels, or medication schedules of a chronically ill patient, when the patient is in between doctor's appointments, can be monitored or guided by a nurse or a care manager.

In some systems such as coordinated care management (CCM) systems, a patient can have, in addition to a physician, a case manager, a care manager, a disease manager, a wellness coach or other health professionals provided by various providers in the system that each seek to manage some or all of the patient's care. However, because these types of health professionals, as well as various doctors and other clinicians, laboratories, and pharmacies may work for different portions of the system and because each professional may not have access to all of the relevant information for caring for a patient, inefficiencies can develop that reduce the effectiveness and increase the cost of patient care.

It would therefore be desirable to provide systems and methods for automating coordinated care management.

### SUMMARY

Systems and methods are provided for automating some or all of a coordinated care management system by mapping medical guidelines to clinical workflows and logistical operations. The medical guidelines may be provided by a healthcare organization such as a hospital, a university, a national or international medical association, or other healthcare organization, or may be derived from a collection of guidelines for similar conditions or circumstances. In particular, medical guidelines are evidence-based guidelines for diagnosis and management and/or treatment for medical conditions. The medical guidelines may be well-established and/or subject to continual update and revision based upon evidence collected by the promulgating organization or entity or other healthcare organizations, including without limitation any local variations due to population demographics, clinical resources or other specific local factors. For example, a medical guideline may be provided for diagnosis and treatment of a condition such as congestive heart failure or a particular bacterial infection. Medical guidelines for a particular bacterial infection may include a guideline that the patient takes a particular antibiotic medication once each day for seven days.

Medical guidelines can be mapped by a system to clinical workflows that provide instructions for action by each participant in a patient care interaction. For example, a clinical workflow may indicate that a doctor is to prescribe an antibiotic and that a patient is to self-administer the antibiotic, or may suggest the location at which health care is to be provided, such as the patient's home, doctor's office, laboratory, or emergency room.

Clinical workflows can be mapped by the system to logistical workflows that determine actions to be taken by each particular participant of a particular healthcare environment in order to implement the clinical workflows. For example, a logistical workflow may be a flowchart that shows each action by each participant including (as examples) sending, from the doctor's office, a prescription to the pharmacy, picking up, by the patient of the prescribed medication, and taking, at specified times by the patient, of the prescribed medication. A clinical workflow may be independent of the particular actors (e.g., the particular patient, the particular physician, the particular pharmacy, the particular laboratory, the particular hospital, the particular care coordinator, etc.) A logistical workflow may be an implementation of the clinical workflow for the particular actors and including, if desired, preferences, habits, and/or actions of the actors. The mapped logistical workflows can be provided in a format that can be automatically translated into computer code.

Logistical workflows may have aspects associated with each group or pool of participants in a healthcare system. A logistical workflow may, for example, include an aspect associated with a patient, an aspect associated with a patient care provider, an aspect associated with a pharmacy, an aspect associated with a laboratory (lab), and/or other aspects or actors. The logistical workflow may include indicators such as communications receipt/transmit indicators, indicators of information exchange between pools or participants, split points at which multiple data are to be obtained, and convergence points at which, unless all data to be obtained has been received, subsequent operations cannot continue.

A system for mapping medical guidelines to clinical workflows and/or logistical workflows and for mapping clinical workflows to logistical workflows may be a fully automated system or one or more human operators may perform one or more mapping, optimizing, programming, designing, correcting, and/or communicating functions for the system.

Each of the logistical and/or clinical workflows may also be mapped and/or remapped based upon information or inputs regarding specific elements of a given clinical environment, such as the skillsets, location, availability and schedule of the various members of a care provider team. In addition, if a clinical workflow or medical guideline is mapped to a logistical workflow and such map results in a logistical constraint that is incompatible with a given patient's or clinical environment's constraints, the system may provide an alert or other communication to a human operator alerting the operator of such incompatible constraint so that it may be resolved by the operator, or the system may automatically resolve such constraint. For example, a clinical workflow for a patient with a bacterial infection may call for such patient to begin a course of antibiotics within 24 hours, and when mapped onto the logistical workflow, the logistical workflow calls for the prescription to be filled at a neighborhood pharmacy; however, the patient is home-bound and has no means of picking up his or her prescription. According to an embodiment, the system would communicate to an operator the existence of the incompatibility of the logistical workflow (onto which the clinical workflow or medical guideline was mapped) with the patient's condition and/or preferences, and the system would potentially remap the logistical workflow to provide for home delivery to the patient of the prescription, and/or alert a system operator to the incompatible constraint.

Systems and methods are provided for generating, by mapping the medical guidelines and clinical workflows into logistical workflows that can be translated into computer code, communications between various portions of a healthcare system to facilitate the coordinated care of patients. Properly deployed, such an integrated and interoperable system could improve a patient's experience with their healthcare system, enhance quality outcomes, improve efficiency and lower overall healthcare delivery costs.

### BRIEF DESCRIPTION OF THE DRAWINGS

FIG. 1 illustrates an example system that can include a mapping system and a communications system for automating portions of a healthcare system in accordance with an embodiment of the disclosure.

FIG. 2 illustrates an example mapping system in accordance with an embodiment of the disclosure.

FIG. 3 illustrates an example communications system in accordance with an embodiment of the disclosure.

FIG. 4 illustrates an example of a mapping process in which medical guidelines are mapped to clinical workflows that are then mapped to logistical workflows that are used to generate communications in accordance with an embodiment of the disclosure.

FIG. 5 illustrates an example of a mapping process in which medical guidelines and existing clinical workflows are mapped to logistical workflows that are used to generate communications in accordance with an embodiment of the disclosure.

FIG. 6 illustrates an example of steps that may be used in automating portions of a healthcare system in accordance with an embodiment of the disclosure.

FIG. 7 illustrates a flow diagram showing how bi-directional communication within the system of FIG. 1 may be provided for updating and optimizing healthcare communications and outcomes in accordance with an embodiment of the disclosure.

FIG. 8 illustrates an example of steps that may be used in updating and optimizing healthcare communications and outcomes in accordance with an embodiment of the disclosure.

FIG. 9 illustrates an example of steps that may be used in remapping medical guidelines and clinical workflows in accordance with an embodiment of the disclosure.

### DETAILED DESCRIPTION

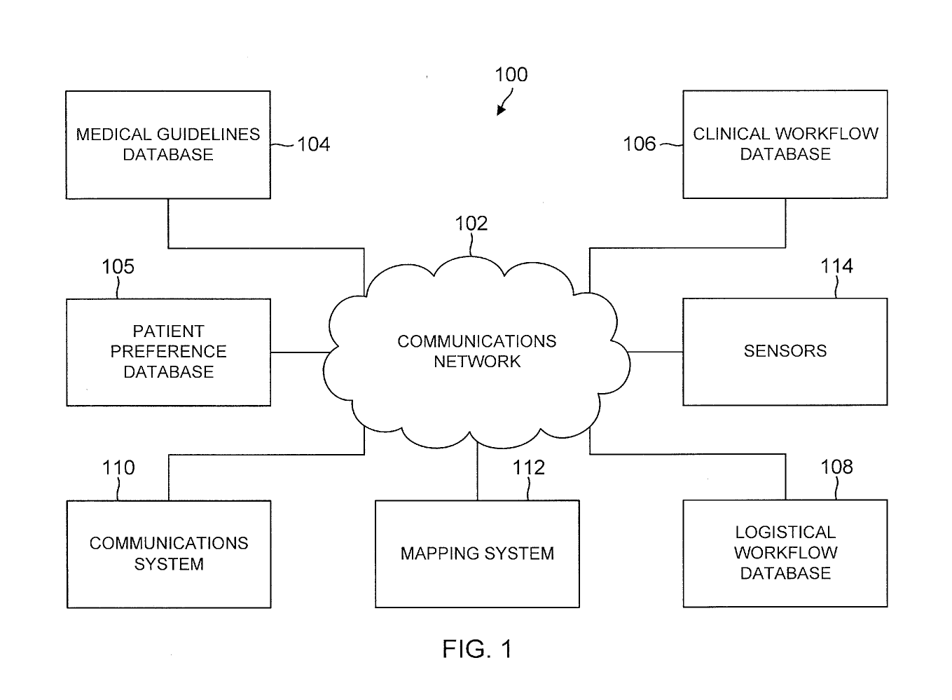

FIG. 1 is a diagram of a system that may be used for generating communications for and between various portions of a healthcare system to facilitate the coordinated care of patients by mapping the medical guidelines and clinical workflows into logistical workflows that can be translated into computer code. As shown in FIG. 1, system 100 may include various components and/or devices that communicate over a network such as communications network 102. System 100 may include one or more databases for storing information such as medical guidelines database 104, clinical workflow database 106, logistical workflow database 108, and patient preference database 105. Databases 104, 105, 106, and 108 may be separate databases (e.g., databases located at various physical locations) or may be portions of a common database. Databases 104, 105, 106, and 108 may be physical installations of memory such as volatile or non-volatile storage media that are located in a common location with other portions of system 100 or may be remote storage installations such as one or more cloud storage servers.

Databases 104, 105, 106, and 108 may be accessed by one or more systems that receive and provide data to the databases such as communications system 110 and mapping system 112.

If desired, one or more sensors 114 may also form a portion of system 100. Sensors 114 may include heart monitors, blood pressure monitors, blood glucose monitors or other automatic or manually controlled sensors for gathering and providing medical information associated with patients into a coordinated care system. In some embodiments, data from sensors 114 may be provided to mapping system 112.

Mapping system 112 may receive medical guidelines from medical guidelines database 104 and map the received medical guidelines to clinical workflows. The clinical workflows may be provided to clinical workflow database 106 for storage. However, this is merely illustrative. If desired, clinical workflows may be provided to clinical workflow database by a third party such as a university or a healthcare or clinical organization. Mapping system 112 may also receive clinical workflows from clinical workflow database 106 and map the clinical workflows to logistical workflows. Mapping system 112 may generate computer code from the logistical workflows. The generated computer code may be provided to communications system 110.

Communications system 110 may generate communications that may have a specific content or tone and which may be in the form of text, email, telephone, social media, or other communications, transactions, and/or notifications. Communications may include a telephone call between an agent and a patient or a care provider, a communication such as a text-based or other message for a patient (e.g., “Remember to pick up your prescription”, “Remember your appointment tomorrow morning”, “Did you visit the lab today?”, etc.), a text-based or other message for a care provider (e.g., “Schedule an appointment for Mr. Smith”, “Follow-up with Ms. Jones regarding her blood glucose measurements”, or “Send the prescription for Ms. Jones to the pharmacy”), a text-based or other message for a pharmacy (e.g., “Fill prescription number A123 for Mr. Smith”), a text-based or other message for a care provider, a text-based or other message for a care coordinator, a text-based or other message for a disease manager, or any other suitable communications for managing patient care, such as an interactive voice recognition system (IVR).

Communications system 110 may, according to some embodiments, accumulate patient response and outcome information such as patient response to communication information (e.g., the time for a patient to respond to various forms of communication such as text messages, emails, and telephone calls) and patient activation information (e.g., the rate at which a patient takes their prescription as a function of the time of day or the form of communication used to remind the patient). Communications system 110 may use accumulated patient response and outcome information to learn (e.g., determine) communications preferences and/or care management preferences for each user that then may become defaults for subsequent communication efficacy. Communications system 110 may use feedback gathered automatically or provided by a patient, a care coordinator or other healthcare system actor back to the communication system in a machine-learning operation to determine the patient preferences.

In various embodiments, any or all of communications system 110, mapping system 112, sensors 114, and databases 104, 105, 106, and/or 108 may be implemented using separate computing equipment (e.g., separate installations of computing equipment at a common location or separate installations of computing equipment at two or more different locations) or may be implemented on common computing equipment.

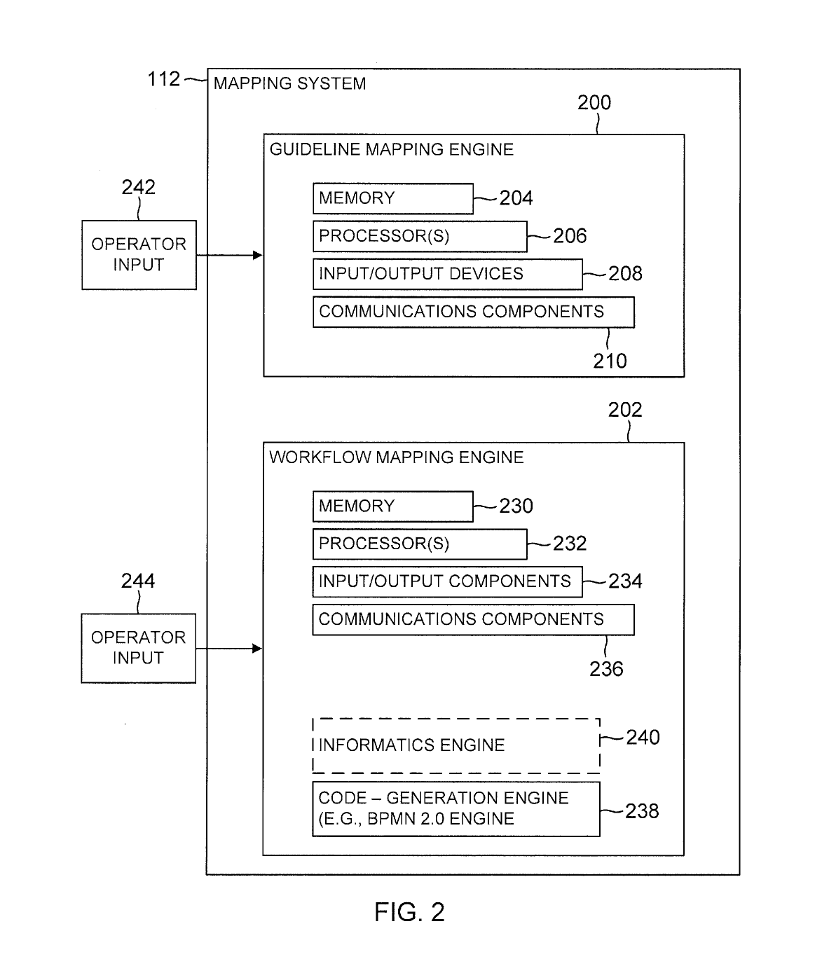

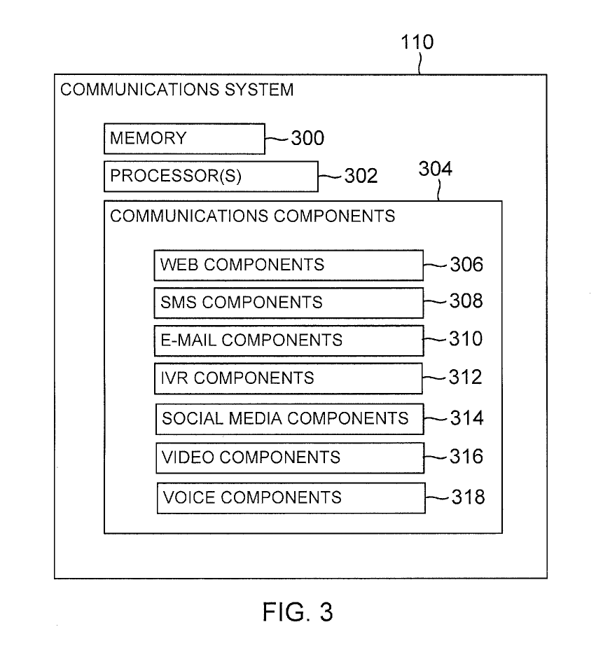

Mapping system 112 and communications system 110 may be implemented using computing equipment as shown respectively in FIGS. 2 and 3. As shown in FIG. 2, mapping system 112 may include a guideline mapping engine 200 and a workflow mapping engine 202. Guideline mapping engine 200 may be used to map medical guidelines to clinical workflows or medical guidelines to logistical workflows. Workflow mapping engine 202 may be used to map clinical workflows to logistical workflows or medical guidelines to logistical workflows.

Guideline mapping engine may include computing equipment such as memory 204, one or more processors 206, input/output devices 208, optional communications equipment 210 and/or other computing equipment suitable for mapping medical guidelines into clinical workflows and/or logistical workflows. Memory 204 may be separate from databases 104, 105, 106, and 108 of FIG. 1 or may include some or all of any of databases 104, 105, 106, 108.

In some embodiments, guideline mapping engine 200 may receive operator input 242 as part of a mapping operation. Operator input may include an electronic copy of a medical guideline (e.g., an operator can scan a paper copy of a medical guideline into an electronic format or otherwise generate or receive an electronic copy of a medical guideline and provide the medical guideline to mapping engine 200), an operator modified medical guideline (e.g., a medical guideline that has been modified by an operator to facilitate machine reading of the medical guideline) or other operator input that facilitates the mapping of a medical guideline into a clinical workflow and/or a logistical workflow.

In some embodiments, an operator may perform some or all of the mapping operations that map a medical guideline into a clinical workflow using, for example, the computing equipment of mapping engine 200. For example, in one embodiment, an operator of mapping engine 200 such as a care coordinator may generate, based upon a medical guideline, a flowchart containing procedural guidelines for members of a healthcare system (e.g., a case manager, a patient, a pharmacist, a laboratory, a patient care provider, a care manager, a care coordinator, a hospital, etc.). A clinical workflow flowchart may include features describing the flow of information such as patient information, test results, analysis, procedures, messaging information, compliance information, or other information, processes or activities involved in care management. A clinical workflow flowchart may include one or more than one decision points. Each decision point may describe, based on one or more inputs, a decision that determines the subsequent flow of information, processes, and/or activities. Decision points may include one or more inputs and an output that is based on the input. The output of a decision point may be based on a relatively simple comparison of data to a standard, comparison of data to other data, or may be based on relatively more complex analysis and optimization operations. As examples, a decision point in a clinical workflow may include an output that is based on an analysis (e.g., a optimization) of a cost such as a patient cost or overall cost to a healthcare system, a safety consideration that optimizes or reduces risk factors for a patient and/or for the system, and/or an efficiency analysis operation that reduces the time for care to be delivered to a patient and/or reduces the time it takes for a care provider, care coordinator, or other member of the healthcare system to deliver and manage care for patients. In this way, decision points in a clinical workflow can be arranged to help increase, manage, or maintain patient safety and/or to optimize costs and increase efficiency of managed care. In one embodiment, the system may include a cost database that can be mapped onto the workflows. In another embodiment, the system may include a previous method effectiveness database based on the history of a specific patient or a population of similar patients that can be mapped onto the workflows.

Workflow mapping engine 202 may include computing equipment such as memory 230, one or more processors 232, input/output components 234, communications components 236 and/or other computing equipment suitable for mapping clinical workflows to logistical workflows. Memory 230 may be separate from memory 204 of guideline mapping engine 200 and databases 104, 105, 106, and 108 of FIG. 1 or may include some or all of any of memory 204 and/or databases 104, 105, 106, and 108.

In some embodiments, workflow mapping engine 202 may receive operator input 244 as part of a mapping operation. Operator input may include an electronic copy of a clinical workflow (e.g., an operator can obtain or generate an electronic copy of a clinical workflow and provide the clinical workflow to workflow mapping engine 202) or other operator input that facilitates the mapping of clinical workflow into a logistical workflow.

An operator may perform some or all of the mapping operations that map a clinical workflow into a logistical workflow using, for example, the computing equipment of workflow mapping engine 202. For example, in one embodiment, an operator of workflow mapping engine 202 may generate, based upon a clinical workflow flowchart (e.g., a flowchart containing procedural guidelines for members of a healthcare system), an additional flowchart that can be provided to a code-generation engine such as code-generation engine 238. The additional flowchart may be a flowchart that is stored in a format that can be converted automatically into machine-readable code.

Code-generation engine 238 may be a software application running on, for example, the computing equipment of workflow mapping engine 202 that automatically generates machine-readable code, which may include without limitation extensible markup language (XML), for causing a computer to generate one or more communications such as notifications. Code-generation engine 238 may, for example, be an implementation of a business process management system (BPMS) package such as a Business Process Model and Notation (BPMN) Version 2.0® application of Object Management Group, Inc. running on the computing equipment of workflow mapping engine 202, communications system 110, or other computing equipment associated with system 100. However, this is merely illustrative. In other embodiments, code-generation engine 238 may be implemented using other software or hardware that generates computer code from a logistical workflow.

If desired, in one embodiment, workflow mapping engine 202 may also include an informatics engine 240 that maps portions of medical guidelines, clinical workflows, and/or logistical workflows to numerical codes that are used by one or more healthcare providers, insurance companies, medical associations, or other portions of a healthcare system. In various embodiments, any or all of code-generation engine 238, optional informatics engine 240, workflow mapping engine 202, and guideline mapping engine 200 may be implemented using separate computing equipment (e.g., separate installations of computing equipment at a common location or separate installations of computing equipment at two or more different locations) or may be implemented on common computing equipment.

FIG. 3 is a diagram of communications system 110 according to an embodiment. As shown in FIG. 3, communications system 110 may include computing equipment such as memory 300, one or more processors 302, and communications components 304. Memory 300 and processor(s) 302 may cooperate to use communications components 304 to generate various communications for patients, doctors, pharmacists, laboratories, care managers, care providers, care coordinators, or other members of a healthcare system. For example, memory 300 may be used to store computer code (machine-readable code) generated by mapping system 112 and processor(s) 302 may be used to execute the stored code. Executing the stored code may cause one or more of communications components 304 to perform communications operations.

Communications components 304 may include components for communicating through various communications mediums such as web components 306, short-message-service (SMS) components 308, e-mail components 310, interactive voice response (IVR) components 312, social media components 314, video components 316, and voice components 318 (e.g., a live operator that performs telephone or other voice communications with patients, care providers, care coordinators and the like). Any or all of web components 306, short-message-service (SMS) components 308, e-mail components 310, interactive voice response (IVR) components 312, social media components 314, video components 316, and voice components 318 can be used to generate particular communications for members of a healthcare system. As examples, notifications such as an appointment-reminder email to a patient, a send-results reminder by telephone to a laboratory, or a patient-follow-up text message reminder to a care coordinator can be generated and transmitted by communications system 110 based on instructions in computer code received from mapping system 112.

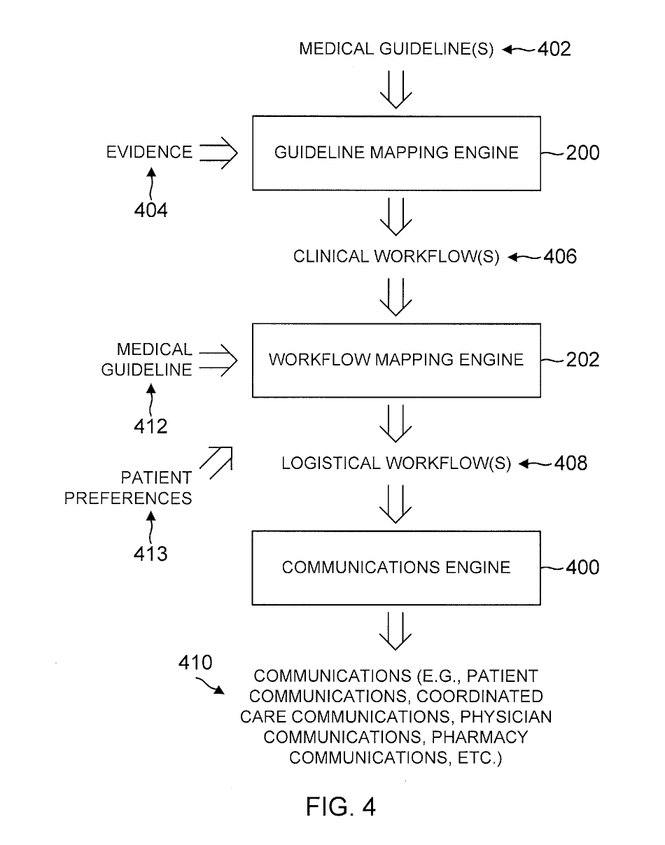

As shown in FIG. 4, one or more medical guidelines 402 may be provided to guideline mapping engine 200. Medical guidelines 402 may be provided to guideline mapping engine automatically or by a human operator (as examples). Guideline mapping engine 200 may generate one or more clinical workflows 406 based on the one or more medical guidelines 402. The clinical workflows may be, for example, procedural guidelines for care providers, patients, and care managers. The clinical workflows may be stored in any suitable format.

In some embodiments, medical or other evidence such as evidence 404 may also be provided to guideline mapping engine 200. Evidence 404 may be used in mapping medical guidelines to clinical workflows and/or logistical workflows. Evidence 404 may include medical evidence or other information such as specific elements of a given clinical environment (e.g., the skillsets, location, availability and/or schedule of one or more members of a care provider team.) For example, a clinical worker that is operating guideline mapping engine 200 may consult medical evidence such as new research publications or other studies that, along with medical guidelines 402, help the operator generate clinical workflow procedures using guideline mapping engine. In another example, the closing of a hospital or the opening of a clinic (as examples) may result in a remapping of a clinical workflow and/or a logistical workflow that accounts for the change in the clinical environment. However, this is merely illustrative. If desired, guideline mapping engine 200 may generate clinical workflows 406 based only on medical guidelines 402. Medical guidelines 402 may be well-established and/or subject to continual update and/or revision based upon evidence collected by the promulgating organization or entity or other healthcare organizations, including not only treatment guidelines, but access to special healthcare competencies or resources or other local conditions.

Clinical workflows 406 may be provided to workflow mapping engine 202. Workflow mapping engine 202 may generate logistical workflows 408 based on the clinical workflows 406. Logistical workflows 408 may be provided to communications engine 400. Communications engine 400 may send and receive communications 410 (e.g., one-way or two-way communications such as patient communications, coordinated care communications, physician communications, pharmacy communications, etc.). According to some embodiments, workflow mapping engine 202 may also receive patient preferences 413 (e.g., from patient preference database 105). Patient preferences 413 may be used to modify and/or optimize logistical workflows 408 (and, in some embodiments, clinical workflows 406). Patient preferences 413 may include patient-provided preferences, learned patient preferences and/or psycho-demographic profiles of a patient or a population of patients (as examples). Communications engine may be implemented using computing equipment of mapping system 112 and/or communications system 110 (e.g., code generation engine 238 and communications components 304).

As shown in FIG. 4, in some embodiments, workflow mapping engine 202 may receive medical guidelines such as medical guidelines 412 and directly generate logistical workflows 408 from the medical guidelines. In another embodiment, guideline mapping engine 200 may receive medical guidelines such as guidelines 402 or 412 and generate logistical workflows 408 directly from the medical guidelines.

In some situations, medical guidelines 402 may be updated and/or revised based upon evidence collected by the promulgating organization or entity or other healthcare organizations, including not only treatment guidelines, but access to special healthcare competencies or resources or other local conditions as described above. For example, an updated version of a previously received medical guideline 402 may be received. The updated version of the medical guideline may then be mapped directly to an updated version of the logistical workflow or to an updated version of the clinical workflow which can then be mapped to an updated version of the logistical workflow. The updated medical guideline can include changes to the medical guideline based on, for example, evidence associated with a population demographic, a clinical resource, and/or other local conditions.

In some situations, a logistical workflow may be generated that includes a conflict with a patient preference or other constraint. For example, a clinical workflow for a patient with a severe burn may call for such patient to be treated at a specialized burn unit, and when mapped onto the logistical workflow, the logistical workflow calls for the patient to be transported via ambulance to the nearest such specialized unit; however, the nearest such specialized unit is filled with patients receiving similar care and is unable to accept the patent at issue. According to an embodiment, the system would remap the logistical workflow to provide for the patient to be transported via air transport to the next closest such specialized unit and/or alert a system operator to the incompatible constraint. In general, any of the steps, processes, or activities described above in connection with FIG. 4 may be performed, rearranged, and/or combined in any order and in any suitable manner for automating coordinated care management.

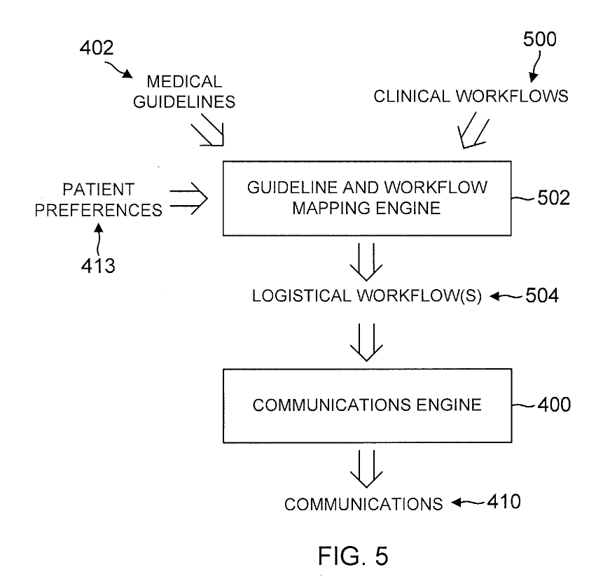

FIG. 5 is a flowchart showing how, in one embodiment, clinical workflows such as clinical workflows 500 may be provided to a guideline and workflow mapping engine 502 that generates logistical workflows 504 from directly provided clinical workflows. Logistical workflows 504 may be provided to communication engine 400. Communication engine 400 may generate communications 410 from workflows 504 as described above in connection with FIG. 4. As shown in FIG. 5, medical guidelines 402 may also be provided to guideline and workflow mapping engine 502 to be mapped to clinical workflows and/or logistical workflows. According to some embodiments, guideline and workflow mapping engine 502 may also receive patient preferences 413 (e.g., from patient preference database 105). Guideline and workflow mapping engine 502 may include various components of guideline mapping engine 200 and/or workflow mapping engine 202 as desired to map medical guidelines 402 to clinical and/or logistical workflows and/or to map clinical guidelines to logistical workflows.

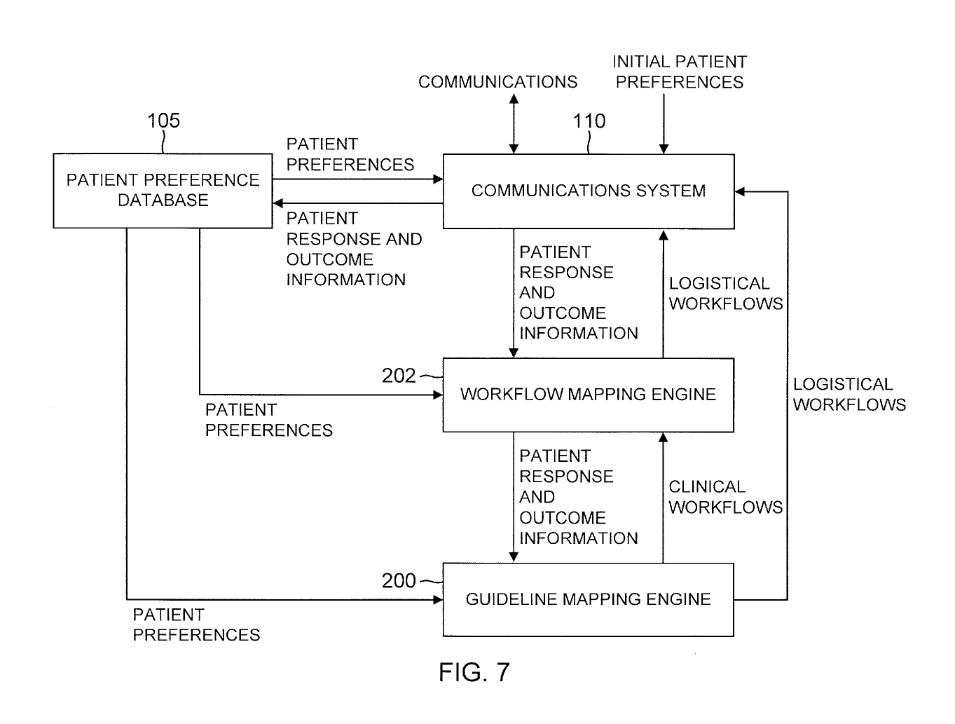

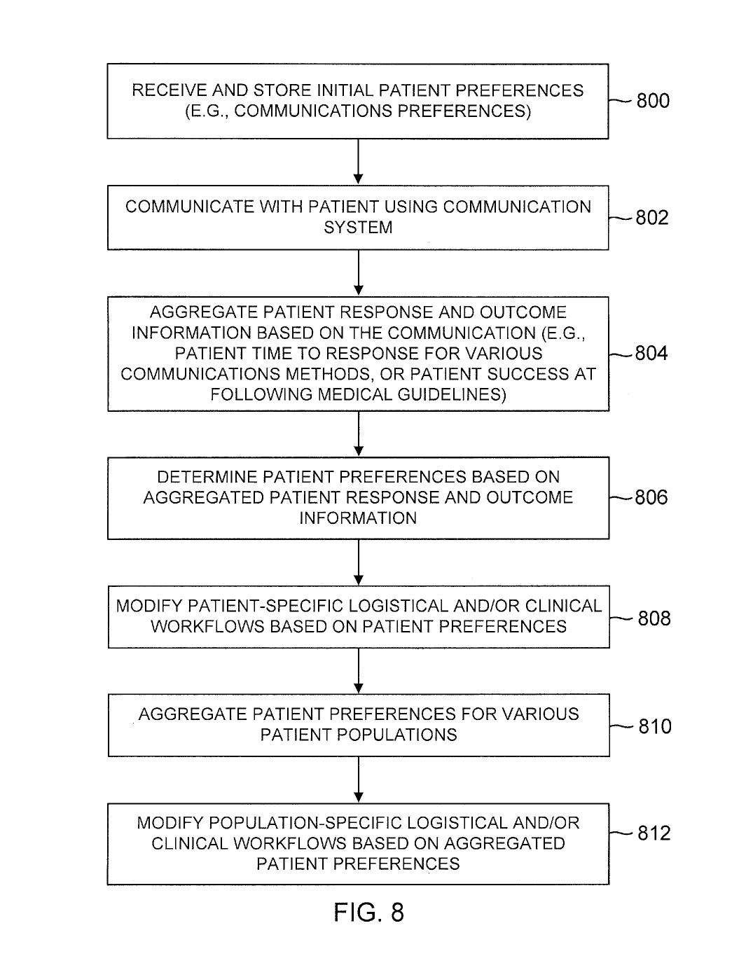

Although the processes described in connection with FIGS. 4 and 5 show unidirectional flow from guideline mapping engine 200 to workflow mapping engine 202, from workflow mapping engine 202 to communications engine 400 and from guidelines and workflow mapping engine 502 to communications engine 400, these systems may, according to various embodiments, have bi-directional communications capabilities. For example, in some embodiments, information such as patient feedback, physician feedback, communications statistical data, or other information may be provided from a patient, physician or other actor to communications engine 400, from communications engine 400 to other portions of system 100 or within system 100. Further details of bi-directional communication within and with system 100 are described hereafter in connection with, for example, FIGS. 7 and 8.

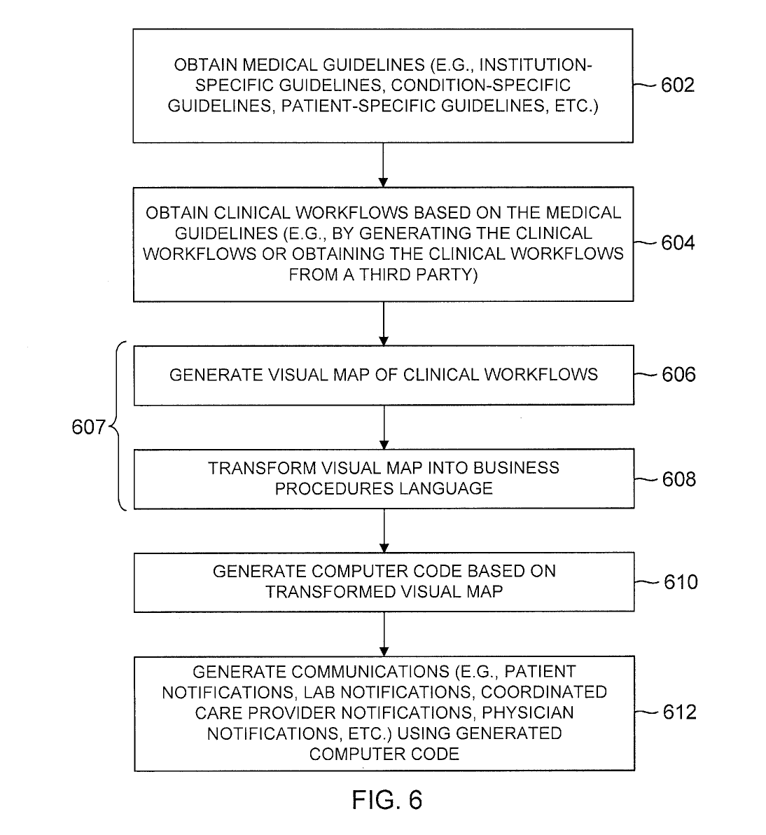

FIG. 6 is a flowchart of illustrative steps that may be performed for automating coordinated care using communications that are automatically generated based on medical guidelines, clinical workflows and/or logistical workflows.

At step 602, medical guidelines may be obtained. Medical guidelines may be obtained from various sources (e.g., universities, hospitals, medical groups, physicians, etc.) and can include institution-specific guidelines, condition-specific guidelines, patient-specific guidelines, or other medical guidelines.

At step 604, clinical workflows may be obtained. The clinical workflows may be generated based on the medical guidelines or obtained from a third-party (as examples). Patient preferences (e.g., patient-specific preferences or population-specific preferences) may also be used, in combination with the medical guidelines, to generate the clinical workflows.

At step 606, a visual map of a clinical workflow may be generated (e.g., a flowchart). A clinical workflow flowchart may include one or more flowcharts describing the flow of information and/or activities associated with patient care.

At step 608, the visual map may be transformed into, for example, a business procedures language such as a business process management system (BPMS) language such as BPMN.

As shown in FIG. 6, the transition between steps 606 and 608 may form a broader step 607 at which a logistical workflow (e.g., the transformed visual map) may be generated from the clinical workflow. The transformed visual map may have any of the various properties of logistical workflows as described herein. The transformed visual map may be an implementation of the clinical workflow flowchart that has been mapped onto the particular clinical environment (e.g., a transformed map that includes particular information specific to the particular patient, the particular physician, the particular pharmacy, the particular laboratory, the particular hospital, the particular care coordinator or other particular actors) and/or the particular habits and/or preferences of the particular actors. Patient preferences (e.g., patient-specific preferences or population-specific preferences) may also be used, in combination with the clinical guidelines to generate logistical workflows.

It should be appreciated that the steps 602, 606, and 608 can be modified to generate the logistical workflow directly from the medical guideline without generating an intervening clinical workflow. For example, a medical guideline such as a renewable prescription may be mapped directly to a logistical workflow such as a particular pharmacy refilling the prescription.

At step 610, computer code (e.g., XML code or other suitable computer code) may be generated based on the transformed visual map.

At step 612, communications may be generated using the generated computer code (e.g., by executing the generated computer code using a memory and one or more processors). The communications may include various one-way, two-way, transactional or other communications. For example, the communications may include notifications such as patient notifications, lab notifications, coordinated care notifications, physician notifications, etc. In another example, the communications may include a transaction such as automatic fulfillment of a prescription, automatic payment for the prescription or other medical services, or an automatic scheduling of an appointment according to various embodiments.

In general, any of the steps, processes, or activities described above in connection with FIG. 6 may be performed, rearranged, and/or combined in any order and in any suitable manner for automating coordinated care management.

FIG. 7 illustrates a flow diagram showing how bi-directional communication within the system of FIG. 1 may be used for updating and optimizing healthcare communications and outcomes. As shown in FIG. 7, communications system 110 may receive initial patient preferences such as preferences for a particular mode of communication (e.g., text messages, personal phone calls, etc.). Communications system 110 may also manage communications with various actors in a healthcare system as described herein. Based on the communications and/or the initial patient preferences, communications system 110 may provide patient response and outcome information to any or all of patient preference database 105, workflow mapping engine 202 and guideline mapping engine 200.

Patient response and outcome information may include information that indicates, for example, the time between a text message, an email, a call, or other communication to a patient by communications system 110 and a response by the patient. Patient response and outcome information may include patient outcome information that indicates that a particular patient takes a prescription at a particular rate following a physical therapy appointment and another particular rate following a text message reminder. Patient response and outcome information may include patient outcome information that indicates the efficacy of a prescription following a physical therapy appointment and/or the efficacy of a prescription following a home visit by a nurse as determined by a physician. However, these examples are merely illustrative. In various embodiments, patient response and outcome information may include any patient provided information, physician provided information, other provided information or automatically gathered information that helps determine patient preferences that can be used to optimize clinical and/or logistical workflows.

Patient response and outcome information may be aggregated in patient preference database 105 and used (e.g., by communications system 110, workflow mapping engine 202, and/or guideline mapping engine 200) to generate patient preferences. Patient preferences in database 105 may be accessible by communications system 110, workflow mapping engine 202, and/or guideline mapping engine 200 and may be used to update initial patient preferences, to optimize communications, to optimize logistical workflows, and/or to optimize clinical workflows.

Patient preferences may be stored in patient preference database 110 and may include patient preference data that indicates, for example, that a particular patient is more responsive to text messages in the morning than text messages in the evening or emails at any time. Patient preferences may indicate that another patient is not responsive to text messages, but a personal phone call to the patient's home that reaches the patient's spouse is effective. Patient preferences may indicate that a particular patient is more likely to take a prescription as directed following a physical therapy appointment or to respond better to the prescription itself following a physical therapy appointment. However, these examples are merely illustrative. In various embodiments, patient preferences may include any patient provided information, physician provided information, other provided information or automatically gathered and/or processed information that helps optimize clinical and/or logistical workflows, reduce costs, increase safety, and/or achieve better patient outcomes.

FIG. 8 is a flowchart of illustrative steps that may be used in obtaining and utilizing patient preferences to optimize patient care communication and outcomes.

At step 800, a system such as system 100 may receive and store initial patient preferences such as patient communications preferences. For example a patient may provide a preference for receiving telephone calls at 8 a.m. on weekdays.

At step 802, the system may communicate with the patient using a communications system such as communications system 110. Communications at step 802 may include, for example, any communications associated with logistical workflows as described herein.

At step 804, the system may aggregate patient response and outcome information based on the communication at step 802. For example, the system may store a patient time-to-response for various communications methods (e.g., text messages, emails, phone calls, video calls, etc.) and/or information indicating a patient's success at following medical guidelines.

At step 806, the system may determine patient preferences as, for example, described herein based on the aggregated patient response and outcome information.

At step 808, the system may modify clinical and/or logistical workflows such as patient-specific clinical and/or logistical workflows based on the aggregated patient response and outcome information. For example, the system may modify a portion of a logistical workflow for a particular patient for a text message reminder at 7 a.m. on weekdays to a text message reminder at 8 a.m. on weekdays.

At step 810, patient preferences or psycho-demographic profiles for various patient populations (e.g., age range populations, gender populations, medical condition populations, geographical populations, ethnicity populations, etc.) may also be aggregated (e.g., in patient preference database 105).

At step 812, population-specific logistical and/or clinical workflows may be modified based on the aggregated patient preferences or psycho-demographic profiles. For example, if patient preferences for one or more single mothers under thirty years old with bachelor's degrees indicates that a particular mode of communication is particularly effective, logistical workflows for other single mothers under thirty years old with bachelor's degrees (e.g., others in the same population) may be modified to preferably use that mode of communication. In another example, if patient preferences for one or more single mothers under thirty years old with bachelor's degrees indicates that a particular drug is particularly effective for that population, clinical workflows may be modified for other single mothers under thirty years old with bachelor's degrees (e.g., others in the same population) to preferably use that drug. However, these examples are merely illustrative. In various embodiments, any aggregated patient preferences may be used to update and/or optimize logistical and/or clinical workflows for any patient population.

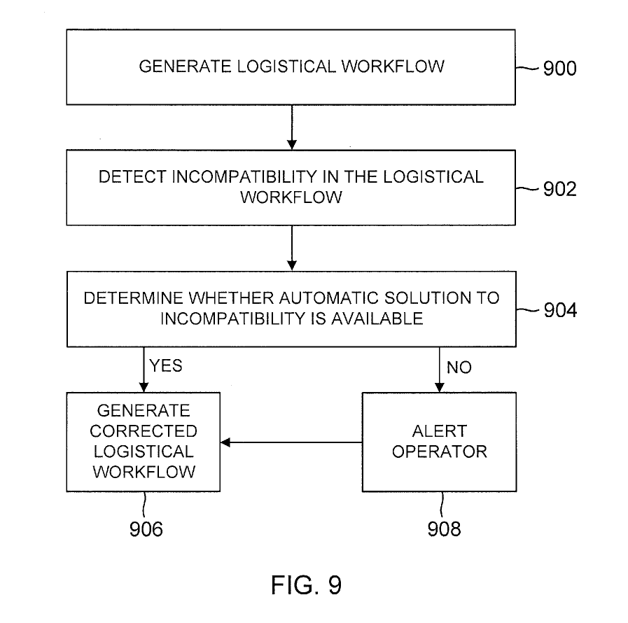

As described herein, in some cases a logistical workflow may include one or more incompatibilities with patient preferences, clinical resources, or other constraints. FIG. 9 is a flowchart of illustrative steps that may be involved in modifying logistical workflows with incompatibilities to generate corrected workflows.

At step 900, a logistical workflow may be generated by a system such as mapping system 112. The logistical workflow may be generated by mapping a clinical workflow to the logistical workflow, mapping a medical guideline directly to the logistical workflow, or mapping a medical guideline to a clinical workflow and then mapping the clinical workflow to the logistical workflow.

At step 902, an incompatibility may be detected in the logistical workflow by the system. For example, the logistical workflow may call for a patient to perform an activity they are unable to physically perform or may call for the patient to be transported to a medical facility that is unable to accept a new patient.

At step 904, the system may determine whether an automatic solution to the incompatibility is available.

In response to determining that an automatic solution is available, the system may proceed to step 906. At step 906, a corrected logistical workflow may be generated. The corrected logistical workflow may be generated by remapping a medical guideline or a clinical workflow to the corrected logistical workflow. The corrected logistical workflow may achieve the desired result from the clinical workflow or the medical guideline without any incompatibilities. For example, the corrected logistical workflow may call for a family member to perform the activity for the patient or may call for the patient to be transported to a different medical facility.

In response to determining that an automatic solution is not available, the system may proceed to step 908. At step 908, an operator such as a system operator may be alerted to the incompatibility. The operator may then provide operator input to the system that allows the system to proceed to step 906, at which a corrected logistical workflow can be generated.

The systems and methods described herein may be combined in any suitable way to provide an integrated and interoperable system for improving a patient's experience with their healthcare system, enhancing quality outcomes, improving efficiency of healthcare management and lowering overall healthcare delivery costs.

The foregoing is merely illustrative of the principles of this invention which can be practiced in other embodiments.

What is claimed is:

1. A mapping system, comprising:
a guideline mapping engine configured to map a medical guideline to at least one of a clinical workflow or a logistical workflow; and
a workflow mapping engine configured to map at least one of the clinical workflow or an additional clinical workflow to an additional logistical workflow.

2. The mapping system defined in claim 1, wherein the guideline mapping engine is configured to map the medical guideline to the logistical workflow.

3. The mapping system defined in claim 1, wherein the guideline mapping engine is configured to map the medical guideline to the clinical workflow and wherein the workflow mapping engine is configured to map the clinical workflow to the additional logistical workflow.

4. The mapping system defined in claim 1, wherein the workflow mapping engine is configured to map the additional clinical workflow to the additional logistical workflow.

5. The mapping system defined in claim 1, wherein the clinical workflow comprises instructions for implementing the medical guidelines and wherein the additional logistical workflow includes user-specific actions based on the clinical workflow.

6. The mapping system defined in claim 1, wherein the medical guideline comprises a document that describes recommendations for diagnosis and management of an illness.

7. The mapping system defined in claim 1, wherein the logistical workflow includes an action to be taken by at least one of a patient, a family member of the patient, a personal care-giver of the patient, or a clinical actor.

8. The mapping system defined in claim 1, wherein the workflow mapping engine comprises a code-generation engine that generates computer code based on the additional logistical workflow.

9. The mapping system defined in claim 8 wherein the computer code comprises code that, when executed on a processor, generates a communication for a user.

10. The mapping system defined in claim 8 wherein the computer code comprises code that, when executed on a processor, executes a transaction.

11. A system, comprising:
a mapping system;
a medical guidelines database;
a clinical workflow database; and
a logistical workflow database, wherein the mapping system is configured to receive medical guidelines from the medical guidelines database, generate clinical workflows to be stored in the clinical workflow database, and generate logistical workflows to be stored in the logistical workflow database.

12. The system defined in claim 11, wherein the mapping system is configured to generate the logistical workflows from the medical guidelines.

13. The system defined in claim 11, wherein the mapping system is configured to generate the logistical workflows from the clinical workflows.

14. The system defined in claim 13, wherein the mapping system is configured to generate the clinical workflows from the medical guidelines.

15. The system defined in claim 11, further comprising:
a communications system that generates communications based on the logistical workflows.

16. The system defined in claim 15, wherein the mapping system comprises a code-generation engine that generates computer code based on the logistical workflows.

17. The system defined in claim 16, wherein the communications system is configured to generate the communications by executing the computer code.

18. The system defined in claim 17, wherein the communications comprise a text message notification to a patient.

19. The system defined in claim 17, wherein the communications comprise an email to a clinical care coordinator.

20. The system defined in claim 17, further comprising a patient preference database and wherein the computer code comprises extensible markup language code.

21. A method, comprising:
receiving a medical guideline from a portion of a healthcare system;
mapping the medical guideline to a clinical workflow; and
mapping the clinical workflow to a logistical workflow.

22. The method defined in claim 21, wherein the medical guideline comprises a document that describes recommendations for diagnosis and management of an illness.

23. The method defined in claim 22, wherein the clinical workflow comprises a flowchart based on the medical guideline.

24. The method defined in claim 23, wherein the logistical workflow comprises Business Process Model and Notation code.

25. The method defined in claim 23, wherein the flowchart comprises at least one decision point.

26. The method defined in claim 25, wherein the at least one decision point includes an output that is based on at least one of a cost, a safety consideration, and a time.

27. The method defined in claim 21, further comprising:
generating computer code based on the logistical workflows; and
generating communications using the generated computer code.

28. The method defined in claim 21, further comprising:
receiving an additional medical guideline; and
mapping the additional medical guideline directly to an additional logistical workflow.

29. The method defined in claim 28, further comprising:
receiving an updated version of the additional medical guideline; and
mapping the updated version of the additional medical guideline directly to an updated version of the additional logistical workflow.

30. The method defined in claim 21, further comprising:
receiving an updated version of the medical guideline;
mapping the updated version of the medical guideline to an updated version of the clinical workflow; and
mapping the updated version of the clinical workflow to an updated version of the logistical workflow, wherein the updated medical guideline includes changes to the medical guideline based on evidence associated with a population demographic and a clinical resource.

31. The method defined in claim 21, further comprising:
detecting an incompatibility in the logistical workflow; and
remapping the clinical workflow to a corrected logistical workflow.

32. A method, comprising:
communicating with a patient using a communications system;
aggregating patient response and outcome information based on the communicating;
determining patient preferences based on the aggregated patient response and outcome information; and
modifying at least one of a clinical workflow or a logistical workflow using the patient preferences.

33. The method defined in claim 32, further comprising:
aggregating the patient preferences for a population of patients; and
modifying at least one of a population-specific clinical workflow or a population-specific logistical workflow using the aggregated patient preferences.

34. The method defined in claim 32, further comprising:
receiving and storing initial patient preferences from the patient.

35. The method defined in claim 34, wherein determining the patient preferences comprises determining the patient preferences by modifying the initial patient preferences.

36. The method defined in claim 32, further comprising:
receiving a medical guideline;
mapping the medical guideline to the clinical workflow; and
mapping the clinical workflow to the logistical workflow.

37. The method defined in claim 32, further comprising:
receiving a medical guideline; and
mapping the medical guideline directly to the logistical workflow.

38. The method defined in claim 32, wherein the clinical workflow comprises a flowchart based on the medical guideline.

39. The method defined in claim 32, wherein the logistical workflow is constructed by a business process management system.

40. The method defined in claim 32, wherein the medical guideline comprises information associated with diagnosis and management of an illness.
